(19) United States
(12) Patent Application Publication
(10) Pub. No.: US 2021/0310885 A1
(43) Pub. Date: Oct. 7, 2021

(54) STRAIN GAUGE ASSEMBLY FORMED OF COMPONENTS HAVING TWO DIFFERENT STIFFNESSES

(71) Applicant: Schaeffler Technologies AG & Co. KG

(72) Inventors: Andrew Dyer, Charlotte, NC (US); Scott Hart, Sharon, SC (US)

(21) Appl. No.: 16/841,073

(22) Filed: Apr. 6, 2020

(51) Int. Cl. G01L 1/22; A61B 34/30

(52) U.S. Cl. CPC G01L 1/2287; A61B 34/30

(57) ABSTRACT

A strain gauge assembly is provided comprising a base including a core and at least one gauge support. The core is longer than the at least one gauge support and the at least one gauge support is fixed to an outer surface of the core. The at least one gauge support has a greater stiffness than the core. The strain gauge assembly also includes at least one strain gauge fixed on an outer surface of the at least one gauge support.

The present disclosure relates generally to strain gauges and more specifically to strain gauges for measuring beam deflection.

### BACKGROUND

A strain gauge can be used to measure or calculate deflection of a beam.

### SUMMARY OF THE INVENTION

A strain gauge assembly is provided comprising a base including a core and at least one gauge support. The core is longer than the at least one gauge support and the at least one gauge support is fixed to an outer surface of the core. The at least one gauge support has a greater stiffness than the core. The strain gauge assembly also includes at least one strain gauge fixed on an outer surface of the at least one gauge support.

Embodiments of the strain gauge assembly may include one or more of the following features alone or in combination. The core may have an elongated shape. The at least one gauge support may be at least one sleeve radially surrounding the core. The core may be a solid cylinder and the at least one sleeve may be a hollow cylinder. The core may be centered on a longitudinal center axis and may be divided into a first half and a second half by a middle plane extending perpendicular to the longitudinal center axis. The at least one gauge support may include a first gauge support fixed to the outer surface of the core on the first half and a second gauge support fixed to the outer surface of the core on the second half. The first gauge support may be closer to a first longitudinal end of the core than to the middle plane and the second gauge support may be closer to a second longitudinal end of the core than to the middle plane. The at least one gauge support may be permanently fixed on the core by press-fit, welding, metal sintering, adhesives or printing. The at least one gauge support may be formed of a first material and the core is formed of a second material. The first material and the second material may be metals. The at least one strain gauge may be at least one sheet fixed to the at least one gauge support or may be deposited on the at least one gauge support via physical vapor deposition.

A medical robot is also provided comprising an actuator and the strain gauge assembly.

A method of constructing a strain gauge assembly is provided comprising providing a core fixing at least one gauge support on an outer surface of the core. The core is longer than the at least one gauge support. The at least one gauge support has a greater stiffness than the core. The method also includes fixing at least one strain gauge on an outer surface of the at least one gauge support.

Embodiments of the method may include one or more of the following features alone or in combination. The core may have an elongated shape and the at least one gauge support may be at least one sleeve radially surrounding the core. The core may be a solid cylinder and the at least one sleeve may be a hollow cylinder. The core may be centered on a longitudinal center axis and the core may be divided into a first half and a second half by a middle plane extending perpendicular to the longitudinal center axis. The at least one gauge support may include a first gauge support and a second gauge support. The fixing the at least one gauge support on the outer surface of the core may include fixing the first gauge support to the outer surface of the core on the first half and fixing the second gauge support to the outer surface of the core on the second half. The first gauge support may be closer to a first longitudinal end of the core than to the middle plane and the second gauge support may be closer to a second longitudinal end of the core than to the middle plane. The fixing of the at least one gauge support on the outer surface of the core may include permanently fixing the at least one gauge support on the core by press-fit, welding, metal sintering, adhesives or printing. The core may be formed of aluminum or steel and the at least one gauge support may be formed of steel. The fixing of the at least one strain gauge on the outer surface of the at least one gauge support may include fixing the at least one strain in the form of at least one sheet to the at least one gauge support or depositing the at least one gauge support on the at least one gauge support via physical vapor deposition. The method may further comprise identifying a location of the core that will experience a highest strain during use of the strain gauge assembly. The fixing of the at least one strain gauge on the outer surface of the at least one gauge support may include fixing a first sleeve of the at least one sleeve on the location that will experience the highest strain during use of the strain gauge assembly.

### BRIEF DESCRIPTION OF THE DRAWINGS

The present disclosure is described below by reference to the following drawings, in which:

FIG. 1 schematically shows a strain gauge assembly in accordance with an embodiment of the present disclosure attached to an actuator of a medical robot;

FIG. 2 shows a radial cross-sectional view of the strain gauge assembly shown in FIG. 1,

FIG. 3 shows a perspective view of the strain gauge assembly shown in FIGS. 2 and 3;

FIG. 4 shows an axial cross-sectional view along A-A in FIG. 2;

FIG. 5 shows a color scale plot illustrating the strain on sleeves of the strain gauge assembly shown in FIGS. 1 to 4.

### DETAILED DESCRIPTION

The disclosure provides strain gauge assembly including a solid core made of a relatively flexible material, for example aluminum, and a sleeve made of relatively stiff material, for example steel. The two different materials of different stiffness make the strain gauge assembly locally stiff to minimize strain at the sensor while the overall assembly is sufficiently flexible. In some embodiments, the strain gauges may be printed on the sleeves.

The strain gauge assembly may be operated by a medical robot including an actuator attached to the end of a strain gauge assembly. The strain gauge assembly can measure force on the actuator by sensing strain on the beam to provide force feedback to a surgeon operating the medical robot. Due to the flexibility of solid core, the strain gauge assembly is flexible and long enough to provide sufficient measurement sensitivity, while the sleeves have a diameter and length that provides sufficient surface area needed for the sensor and is stiff enough to accommodate the maximum strain of the sensors.

The strain gauge assembly of the present disclosure can prevent erroneous deflection calculations caused by permanent deformation of the surface to which the strain gauge is mounted. The strain measurement values are decoupled from the overall beam stiffness such that overall elastic behavior and localized strain can be designed independently.

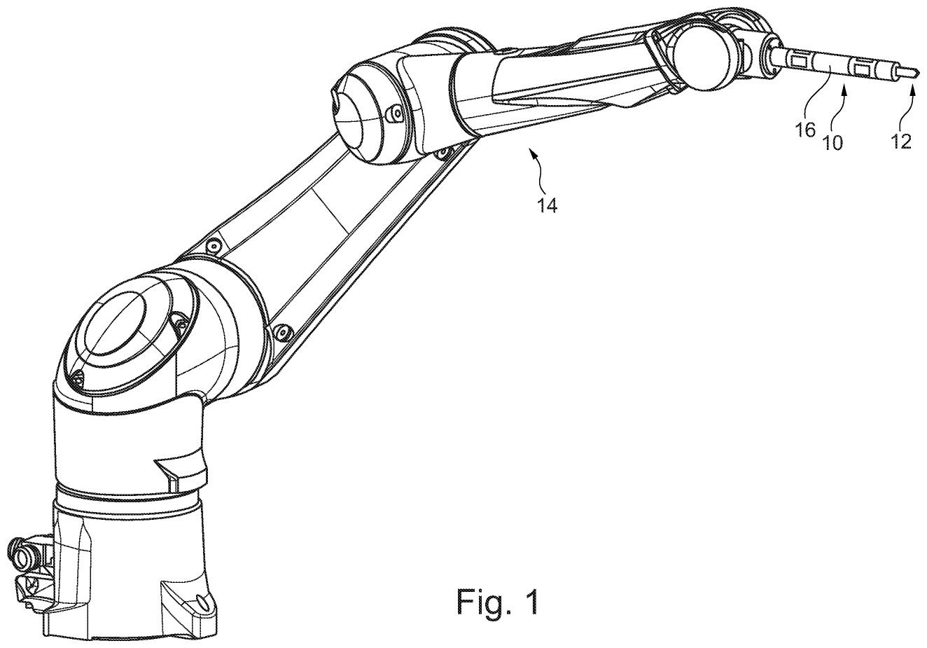

FIG. 1 shows a strain gauge assembly 10 attaching an actuator 12 to a medical robot 14. Actuator 12 is attached to a first longitudinal end of a core 16 of strain gauge assembly 10 and a second longitudinal end of core 16 is attached to robot 14. Strain gauge assembly 10 measures the force on actuator 12 by sensing strain on core 16 to provide force feedback to the surgeon.

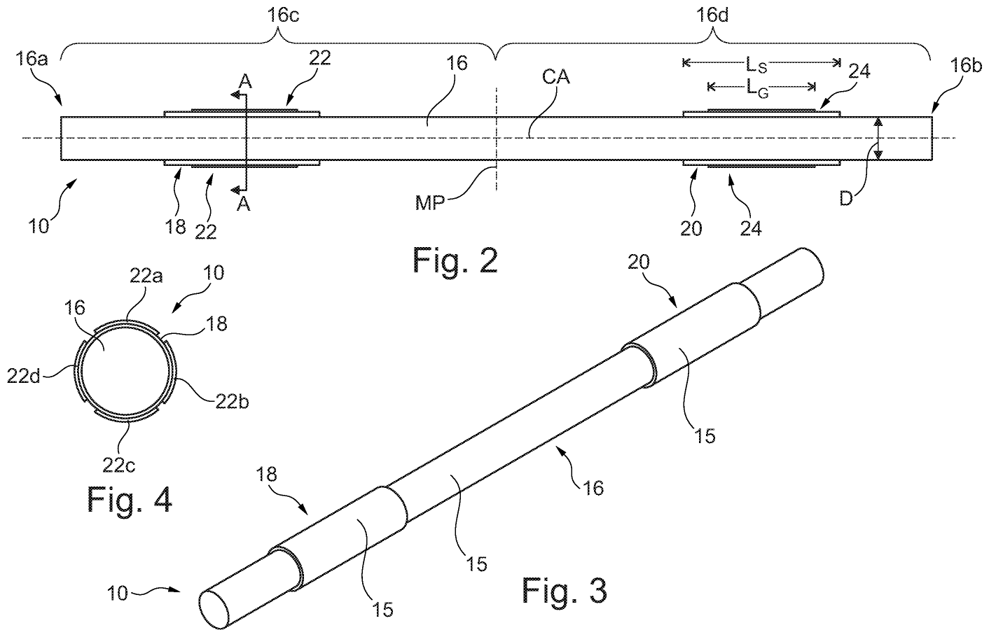

FIGS. 2 and 3 show a cross-sectional view and a perspective view, respectively, of strain gauge assembly 10. Strain gauge assembly 10 includes body 15 formed by elongated core 16 and two gauge supports in the form of sleeves 18, 20 fixed to elongated core 16, and strain gauges 22, 24 (omitted in FIG. 3) fixed to each of sleeves 18, 20, respectively. In other embodiments, only a single sleeve and corresponding strain gauges may be fixed to body 15, or more than two sleeves, each with corresponding strain gauges, may be fixed to body 15. In the embodiment shown in FIGS. 2 and 3, elongated core 16 is a solid cylindrical beam and sleeves 18, 20 are hollow cylinders fixed to an outer circumferential surface of core 16 and radially surrounding core 16. Elongated core 16 is larger than sleeves 18, 20 in volume, mass and length. In other embodiments, core 16 may be elongated with a polygonal cross-section, for example square or hexagonal, with sleeves 18, 20 having a same corresponding polygonal cross-section.

As shown in FIG. 4, which is a view along A-A in FIG. 2, sleeve 18 is provided with four strain gauges 22, denoted 22a, 22b, 22c, 22d in FIG. 4. Gauges 22a to 22d are circumferentially spaced from each other such that gauges 22a and 22c are directly opposite of each other and gauges 22b and 22d are directly opposite of each other. In another embodiment, one set of opposite gauges—i.e., either gauges 22a and 22c or gauges 22b and 22d may be omitted.

Core 16 extends longitudinally from a first longitudinal end 16a to a second longitudinal end 16b and is centered on a longitudinal center axis A. The terms radial, axial, circumferential and derivatives thereof are used herein in reference to center axis CA, unless otherwise specified. A middle plane MP extends perpendicular to center axis CA halfway between ends 16a, 16b. Middle plane MP divides core 16 into a first longitudinal half 16c including first end 16a and a second longitudinal half 16d including second end 16b.

First sleeve 18 is fixed on a first half 16c of core 16 and second sleeve 20 is fixed on a second half 16d of core 16 such that sleeves 18, 20 are spaced apart from each other. Each of sleeves 18, 20 cover less than half of the outer circumferential surface of the respective half 16c, 16d of the core 16. Each of sleeves 18, 20 is closer to the respective end 16a, 16b than the middle plane MP. Sleeves 18, 20 may be permanently fixed on core 16 by for example press-fit, welding, metal sintering, adhesives or printing. Sleeves 18, 20 are fixed on core 16 in a manner such that strain experienced by core 16 is transferred from sleeves 18, 20 to the respective strain gauge 22, 24 on sleeves 18, 20. Sleeves 18, 20 are fixed to core 16 such that the inner circumference of each of sleeves 18, 20 abuts the outer circumference of core 16 and sleeves 18, 20 are mounted concentric to center axis CA.

In one embodiment, each of gauges 22, 24 has a length L_{G} that is equal to or greater than the length L_{S} of the corresponding sleeve 18, 20 minus ⅔ of the diameter D of the core 16, with the upper limit of the length L_{G} of each strain gauge 22, 24 being the length L_{S} of the corresponding sleeve 18, 20.

Strain gauges 22, 24 are fixed directly on the outer circumferential surfaces of respective sleeves 18, 20. In one embodiment, strain gauges 22, 24 may be formed as a sheet and each include a carrier layer made of plastic that is fixed to the outer circumference of the respective sleeve 18, 20 by adhesive and an electrical circuit on the carrier layer. In another embodiment, the strain gauges 22, 24 may be formed by depositing materials directly onto the outer circumferential surfaces of the respective sleeves 18, 20 for example using Schaeffler's Sensotect coasting technology. A strain sensitive alloy may be deposited for example by physical vapor deposition (PVD), then structured via a laser to form circuitry. Next, a mask can be applied, and electrical contacts pads can be deposited onto the surface.

Core 16 and sleeves 18, 20 are formed of different materials. Core 16 has a first stiffness and sleeves 18, 20 have a second stiffness that is greater than the first stiffness such core 16 is more flexible than sleeves 18, 20. The parameter for measuring stiffness is Young's Modulus. Core 16 may be made of a single material and sleeves 18, 20 may each be made of a same single material such that core is made of a first material and sleeves 18, 20 are made of a second material. The first material and the second material may be metals. In one embodiment, core 16 may be formed of aluminum and sleeves 18, 20 may be formed of steel. In another embodiment, core 16 may be formed of a first steel, for example stainless steel, and sleeves 18, 20 may be formed of a second steel, for example carbon steel, having a higher Young's Modulus than the first steel. Core 16 is more sensitive to strain due to its flexibility relative to the sleeves 18, 20, and the sleeves 18, 20 are each more resistant to forces that could damage the strain gauges supported thereon. In other embodiments, core 16 may be made of two or more materials and sleeves 18, 20 may each be made of two or more materials,

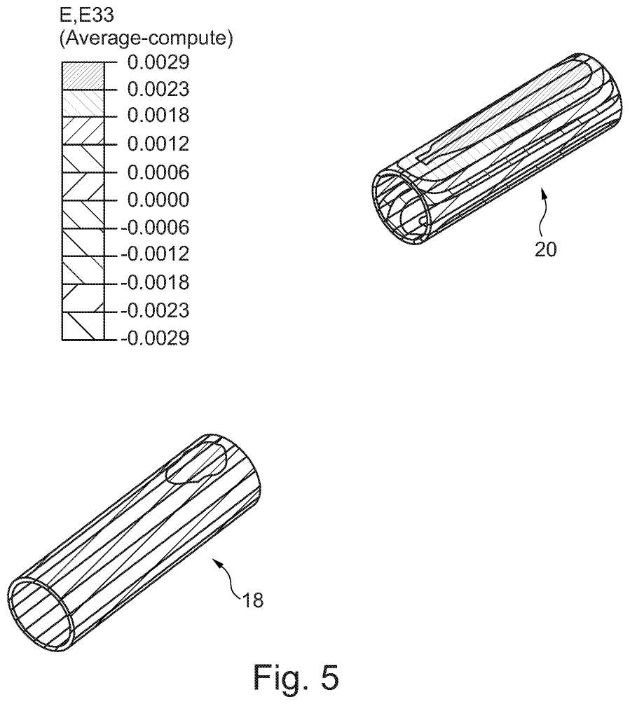

FIG. 5 shows a color scale plot illustrating the strain on each of sleeves 18, 20, illustrating that sleeve 20 is experiencing a much greater strain than sleeve 18. A method of the present disclosure may include identifying a location of core 16 that will experience the highest strain during use of the strain gauge assembly 10—i.e., the location of the core 16 most sensitive to bending, then positioning sleeve 20 on the location that will experience the highest strain, and positioning sleeve 18 on a location that will experience less strain than sleeve 20 during use of strain gauge assembly 10. Mathematics may then be used to interpolate and extrapolate the conditions causing the beam to respond in the manner the gauges are reporting.

In another exemplary embodiment, a single sleeve may be positioned on the location of the beam most sensitive to bending and this single location may be used to determine the response of the beam.

The strain on base 15 is measured based on the material properties of sleeves 18, 20 such that the behavior can be tailored to a range of values targeted. The material properties of core 16 control the overall stiffness/deflection behavior of base 15 and permits a different elastic behavior of base 15 to occur compared with the elastic behavior of a beam based only on the material in sleeves 18, 20 or the material in core 16.

In the preceding specification, the disclosure has been described with reference to specific exemplary embodiments and examples thereof. It will, however, be evident that various modifications and changes may be made thereto without departing from the broader spirit and scope of disclosure as set forth in the claims that follow. The specification and drawings are accordingly to be regarded in an illustrative manner rather than a restrictive sense.

### LIST OF REFERENCE NUMERALS

CA center axis
D core diameter
L_{G} gauge length
L_{S} sleeve length
MP middle plane
10 strain gauge assembly
12 actuator
14 medical robot
15 body
16 core
16a, 16b core longitudinal ends
16c, 16d core halves
18, 20 sleeves
22,22a, 22b, 22c, 22d, 24 strain gauges

What is claimed is:

1. A strain gauge assembly comprising:
a base including a core and at least one gauge support, the core being greater in length than the at least one gauge support, the at least one gauge support being fixed to an outer surface of the core, the at least one gauge support having a greater stiffness than the core; and
at least one strain gauge fixed on an outer surface of the at least one gauge support.

2. The strain gauge assembly as recited in claim 1 wherein the core has an elongated shape.

3. The strain gauge assembly as recited in claim 2 wherein the at least one gauge support is at least one sleeve radially surrounding the core.

4. The strain gauge assembly as recited in claim 3 wherein the core is a solid cylinder and the at least one sleeve is a hollow cylinder.

5. The strain gauge assembly as recited in claim 2 wherein the core is centered on a longitudinal center axis and the core is divided into a first half and a second half by a middle plane extending perpendicular to the longitudinal center axis, the at least one gauge support including a first gauge support fixed to the outer surface of the core on the first half and a second gauge support fixed to the outer surface of the core on the second half.

6. The strain gauge assembly as recited in claim 5 wherein the first gauge support is closer to a first longitudinal end of the core than to the middle plane and the second gauge support is closer to a second longitudinal end of the core than to the middle plane.

7. The strain gauge assembly as recited in claim 1 wherein the at least one gauge support is permanently fixed on the core by press-fit, welding, metal sintering, adhesives or printing.

8. The strain gauge assembly as recited in claim 1 wherein the at least one gauge support is formed of a first material and the core is formed of a second material.

9. The strain gauge assembly as recited in claim 8 wherein the first material and the second material are metals.

10. The strain gauge assembly as recited in claim 1 wherein the at least one strain gauge is at least one sheet fixed to the at least one gauge support or is deposited on the at least one gauge support via physical vapor deposition.

11. A medical robot comprising:
an actuator; and
the strain gauge assembly as recited in claim 1 fixed to the actuator.

12. A method of constructing a strain gauge assembly comprising:
providing a core;
fixing at least one gauge support on an outer surface of the core, the core being of a greater length than the at least one gauge support, the at least one gauge support having a greater stiffness than the core; and
fixing at least one strain gauge on an outer surface of the at least one gauge support.

13. The method as recited in claim 12 wherein the core has an elongated shape and the at least one gauge support is at least one sleeve radially surrounding the core.

14. The method as recited in claim 13 wherein the core is a solid cylinder and the at least one sleeve is a hollow cylinder.

15. The method as recited in claim 14 wherein the core is centered on a longitudinal center axis and the core is divided into a first half and a second half by a middle plane extending perpendicular to the longitudinal center axis,
the at least one gauge support including a first gauge support and a second gauge support,
the fixing the at least one gauge support on the outer surface of the core including fixing the first gauge support to the outer surface of the core on the first half and fixing the second gauge support to the outer surface of the core on the second half.

16. The method as recited in claim 15 wherein the first gauge support is closer to a first longitudinal end of the core than to the middle plane and the second gauge support is closer to a second longitudinal end of the core than to the middle plane.

17. The strain gauge assembly as recited in claim 12 wherein the fixing of the at least one gauge support on the outer surface of the core includes permanently fixing the at least one gauge support on the core by press-fit, welding, metal sintering, adhesives or printing.

18. The method as recited in claim 12 wherein the core is formed of aluminum or steel and the at least one gauge support is formed of steel.

19. The method as recited in claim 12 wherein the fixing of the at least one strain gauge on the outer surface of the at least one gauge support includes fixing the at least one strain in the form of at least one sheet to the at least one gauge support or depositing the at least one gauge support on the at least one gauge support via physical vapor deposition.

20. The method as recited in claim 12 further comprising identifying a location of the core that will experience a highest strain during use of the strain gauge assembly,
the fixing of the at least one strain gauge on the outer surface of the at least one gauge support including fixing a first sleeve of the at least one sleeve on the location that will experience the highest strain during use of the strain gauge assembly.
